(19) United States
(12) Patent Application Publication
(10) Pub. No.: US 2023/0119081 A1
(43) Pub. Date: Apr. 20, 2023

(54) DRIVE MODULES FOR INJECTORS, AND SYSTEMS AND METHODS FOR USING SUCH DRIVE MODULES

(71) Applicant: ALTAVIZ, LLC, Irvine, CA (US)

(72) Inventors: Matthew McCawley, San Clemente, CA (US); Jack R. Auld, Laguna Niguel, CA (US)

(21) Appl. No.: 17/965,707

(22) Filed: Oct. 13, 2022

Related U.S. Application Data
(60) Provisional application No. 63/255,884, filed on Oct. 14, 2021.

(51) Int. Cl. A61M 5/20; A61M 5/315; A61M 5/32

(52) U.S. Cl. CPC A61M 5/2046; A61M 5/3204; A61M 5/31511; A61M 2005/208

(57) ABSTRACT

An injection device for delivering an agent into a patient’s body including a drive module and an injector module. The drive module includes a canister and puncture mechanism in a first chamber, a plunger in a second chamber communicating with the first chamber, and an actuator that moves the puncture mechanism to cause a puncture pin thereon to penetrate a septum of the canister and cause the gas within the canister to flow through the first chamber into the second chamber. The plunger includes a proximal end including a piston and a distal end and is configured such that gas entering the second chamber causes the plunger to move from an initial position to an extended position for delivering one or more agents from the injector module, the piston including one or more passages that allow gas from the canister to pass through the piston into the second chamber around the plunger.

### RELATED APPLICATION DATA

The present application claims benefit of co-pending U.S. Provisional Application Serial No. 63/255,884, filed Oct. 14, 2021, the entire disclosure of which is expressly incorporated by reference herein.

### TECHNICAL FIELD

The present application relates generally to devices and methods for delivering agents into a subject’s body and, more particularly, to auto-injectors and/or gas-powered drive systems for injection devices, and to methods for making and using such devices.

### BACKGROUND

There are many applications involving delivery of a medicament or other agent subcutaneously, intramuscularly, or otherwise into a patient’s body. For example, auto-injectors are available that include a predetermined dose of the agent that may be delivered automatically into the patient’s body, e.g., after placement against the patient’s skin and activation. Generally, such auto-injectors are spring-loaded syringes that are activated to release the spring, which generates sufficient force to penetrate the skin with a needle and deliver the dose within the syringe. For viscous fluids, the forces required to develop fluid flow can be higher than spring-powered systems can provide. When springs can be used, they must generate a relatively high force that requires springs of high mass. Consequently, such auto-injectors may make substantial noise, create pressure spikes in the syringe leading to glass breakage, vibrate, and/or may drive the needle forcefully into the patient’s skin, which may cause pain and/or may startle the user, particularly when the patient is administering the injection themselves.

Therefore, improved devices and methods for delivering agents into a patient’s body would be useful.

### SUMMARY

The present application relates generally to devices and methods for delivering agents into a subject’s body and, more particularly, to auto-injectors and/or gas-powered drive systems for injection devices, and to methods for making and using such devices.

In accordance with one example, a drive module is provided for an injection device for delivering one or more agents into a subject’s body that includes an elongate drive housing including a first end and a second end, and a chamber; a source of pressurized gas communicating with a passage into the chamber; a plunger slidably disposed within the chamber comprising a proximal end including a piston and a distal end, the plunger configured such that gas entering the chamber from the passage causes the plunger to move from an initial position to an extended position wherein the distal end of the plunger extends from the second end of the drive housing for delivering one or more agents from an injector module based on movement of the plunger, wherein the piston comprises one or more passages configured to allow gas from the canister entering the chamber to pass through the piston into the chamber around the plunger. Optionally, the plunger may include a plunger chamber extending from an opening in the proximal end communicating with the second such that pressurized gas from the canister entering the chamber fills the plunger chamber.

In accordance with another example, a device is provided for delivering one or more agents into a patient’s body that includes a) a drive module including an elongate drive housing including a first end and a second end, a first chamber adjacent the first end communicating with a second chamber adjacent the second end via an intermediate passage; a puncture mechanism within the first chamber adjacent the first end including a puncture pin; a canister containing pressurized gas including a penetrable septum disposed adjacent the puncture pin; an actuator configured to move one of the puncture mechanism and the canister to cause the puncture pin to penetrate the septum and cause the gas within the canister to flow through the first chamber around the canister, through the intermediate passage, and into the second chamber; and a plunger slidably disposed within the second chamber comprising a proximal end including a piston and a distal end, the plunger configured such that gas entering the second chamber from the passage causes the plunger to move from an initial position to an extended position wherein the distal end of the plunger extends from the second end of the drive housing for delivering one or more agents from an injector module based on movement of the plunger. The device also includes an injector module including an injector housing coupled to the drive housing carrying an agent chamber containing one or more agents; a piston slidably disposed within the agent chamber and coupled to the distal end of the plunger; and a needle extending from the injector module opposite the drive housing and communicating with the agent chamber for delivering the one or more agents from the agent chamber when the plunger moves from the retracted position to the extended position, thereby advancing the piston within the agent chamber, wherein the piston comprises one or more passages configured to allow gas from the canister entering the first chamber to pass through the piston into the chamber around the plunger.

The devices herein may be used to perform an injection, e.g., a method including inserting the needle through a patient’s skin; and activating an actuator to deliver pressurized gas from the source through the passage into the chamber, thereby moving the plunger from the initial position to the extended position and, consequently, advancing the piston within the agent chamber to deliver the one or more agents through the needle into the patient’s body.

Other aspects and features of the present invention will become apparent from consideration of the following description taken in conjunction with the accompanying drawings.

### BRIEF DESCRIPTION OF THE DRAWINGS

The invention is best understood from the following detailed description when read in conjunction with the accompanying drawings. It is emphasized that, according to common practice, the various features and design elements of the drawings are not to-scale. On the contrary, the dimensions of the various features and design elements are arbitrarily expanded or reduced for clarity. Included in the drawings are the following figures.

FIG. 1 is a side view of an exemplary auto-injector device.

FIGS. 2A and 2B are cross-sectional views of the auto-injector device of FIG. 1 taken along plane A-A and showing details of a gas-powered drive module and an injector module.

FIGS. 3A and 3B are cross-sectional views of an example of a gas-powered drive module that may be included in the device of FIGS. 1-2B, showing the drive module before and after activation, respectively.

FIGS. 4A and 4B are cross-sectional side views of an alternative example of a gas-powered driver for an injector, similar to the drive module shown in FIGS. 3A and 3B but with the orientation of the canister and pin mechanism reversed.

FIGS. 5A and 5B are cross-sectional side views of a syringe driver that minimizes force drop during advancement of a plunger of the driver.

### DETAILED DESCRIPTION

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

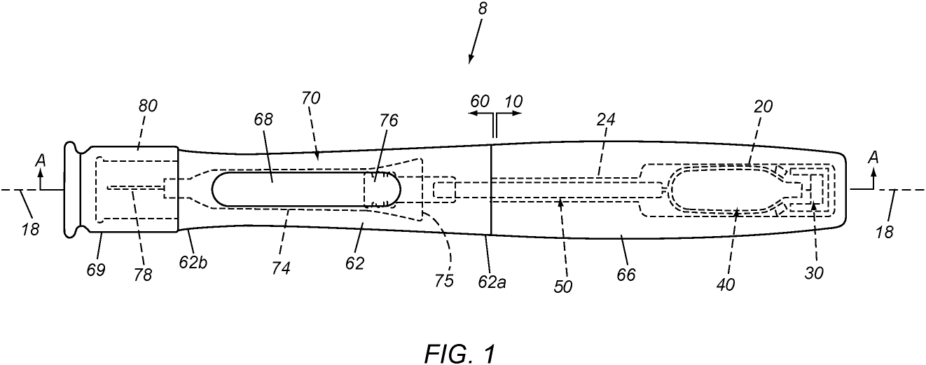

Turning to the drawings, FIGS. 1-3B show an example of an injection device 8 that includes a gas-powered drive cartridge or module 10 and an injector cartridge or module 60 coupled to the drive module 10, which may include components and/or perform similar to any of the examples described herein. Generally, the device 8 may be an auto-injector with the drive module 10 providing force or power that, upon activation, automatically delivers one or more agents contained within the injector module 60, e.g., similar to the injectors disclosed in U.S. Pat. No. 11,071,824, the entire disclosure of which is expressly incorporated by reference herein as described elsewhere herein. As used herein, “agent” may include one or more therapeutic and/or diagnostic compounds or materials, e.g., in liquid or gaseous form, in solution or suspension, and the like, such as viscous fluids.

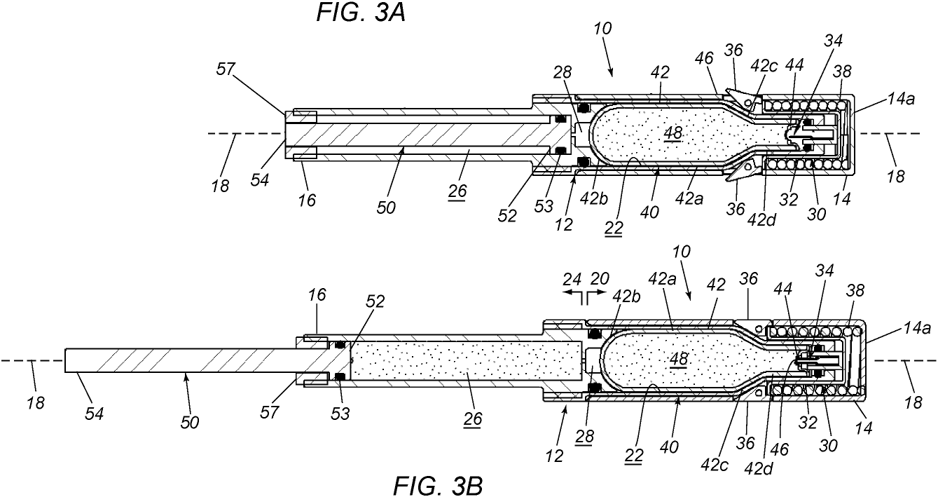

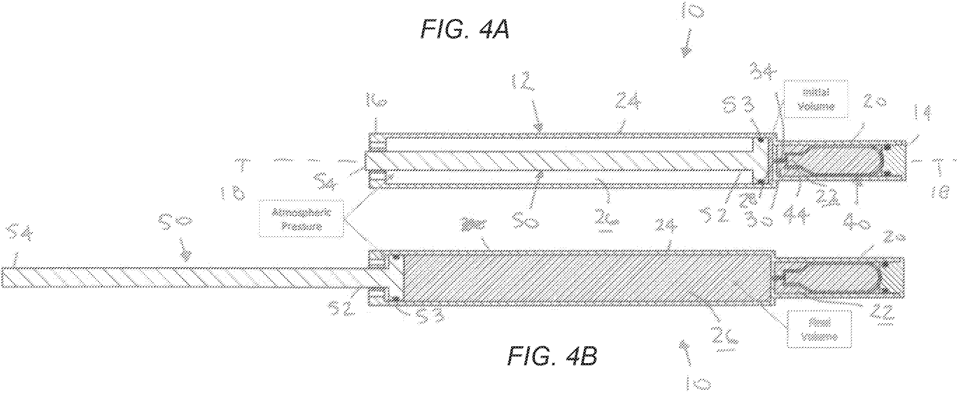

FIGS. 3A and 3B show an example of a drive module 10 that may be included in the auto-injector 8 (or other injection device) used to deliver one or more agents into a subject’s body. FIGS. 4A and 4B show the drive module 10 with the canister 40 and pin mechanism 30 reversed, but otherwise operating similarly. In one example, the drive module 10 may be integrated into or coupled to a syringe module or other injector device that contains an agent.

Generally, the drive module 10 includes an elongate drive housing 12 containing a puncture mechanism 30 including a puncture pin 34, a gas canister or other source of pressurized gas 40, and a plunger 50. The housing 12 includes an enclosed first or proximal end 14 and an open second or distal end 16 aligned along a longitudinal axis 18. The housing 12 may be formed as a single, integral component, e.g., from metal, such as steel, aluminum, and the like, plastic, and/or composite material, by one or more of cold drawing, molding, casting, machining, and the like. Alternatively, the housing 12 may be formed from multiple, separate components that are substantially permanently attached together, e.g., by one or more of welding, soldering, fusing, bonding with adhesive, interference fit, and the like.

The housing 12 includes a first or proximal portion 20 adjacent the first end 14 defining a first chamber 22 for receiving the canister 40 and puncture mechanism 30, and a second or distal portion 24 adjacent the second end 16 defining a second chamber 26 communicating with the first chamber 22 via an intermediate passage 28. Both housing portions 20, 24 may have a generally cylindrical or other appropriate shape, e.g., defining a substantially uniform outer and/or inner diameter.

As shown, the puncture mechanism 30 may be provided within the first chamber 22 adjacent the intermediate passage 28 and the canister 40 may be located proximal to the puncture mechanism 30 with a septum 44 of the canister 40 spaced apart initially from the puncture pin 34. Alternatively, the puncture mechanism 30 may be provided within the first chamber 22 immediately adjacent the first end 14 and the canister 40 containing compressed gas may be disposed within the first chamber 22 adjacent the puncture mechanism 30, e.g., again with the puncture pin 34 initially spaced apart from the septum 44.

Generally, with particular reference to FIGS. 3A and 3B, the canister 40 includes a body 42 and a cap 44 including a septum 46 welded to the body 42 to provide an enclosed cavity 48 filled with a fluid containing liquefied gas, such as carbon dioxide or fluorocarbon gases, compressed to sufficient pressure to least partially liquefy the gas within the cavity 48. Alternatively, fluids containing gases such as argon, nitrogen, helium argon, or other combinations thereof that remain in gaseous form may be stored within the cavity 48. As described elsewhere herein, the fluid contained within the cavity 48 may be used to provide a desired potential energy or discharge force to drive the injection device 8, e.g., to inject one or more agents from the injector module 60 into a patient’s body. In one example, the body 42 and cap 44 may be formed from stainless steel or other desired or suitable metal, plastic, or composite material, e.g., formed by one or more of drawing, stamping, machining, casting, molding, and the like. For example, the body 42 may be deep drawn from sheet metal, e.g., a round sheet metal blank of Type 305 stainless steel, using one or more dies and punches (not shown), to form a main barrel region 42a, an enclosed base or first end 42b, a tapered shoulder region 42c, and an open neck region or second end 42d defining an opening or passage within which the cap 44 is attached. Additional information regarding canisters that may be used and methods for making them may be found in U.S. Publication No. 2017/ 0258583, the entire disclosure of which is expressly incorporated by reference herein.

In the example shown in FIGS. 3A and 3B, the puncture mechanism 30 includes a pin sleeve 32 slidably disposed around the first chamber 22 adjacent the first end 14, i.e., between the second end 42d and cap 44 of the canister 40 and a proximal wall 14a and/or sealed by proximal and distal O-rings enclosing the first chamber 22. The pin sleeve 32 carries a puncture pin 34 and is movable within the first chamber 22, e.g., axially between an inactive position wherein the puncture pin 34 is spaced apart from the septum 46 (FIG. 3A) and an active position wherein the puncture pin 34 penetrates the septum 46 (FIG. 3B) to release gas from the cavity 48 of the canister 40, as described elsewhere herein.

The pin sleeve 32 may be biased to the active position and restrained in the inactive position, e.g., by one or more catches 36 on the drive housing 12 restraining the pin sleeve 32 in the inactive position. For example, a compression spring 38 may be disposed around the pin sleeve 32 and/or otherwise coupled between the housing 12 and the pin sleeve 32 to direct the pin sleeve 32 from the inactive position to the active position when activated.

For example, as shown in FIGS. 3A and 3B, the spring 38 may be constrained between a distal flange 32a on the pin sleeve 32 and the proximal wall 14a of the drive housing 12. A pair of catches 36 may be mounted to the drive housing 12 that are pivotable from an outward orientation (FIG. 3A), where the catches 36 contact the distal flange 32a to prevent movement of the pin sleeve 32 from the inactive position, to an inward position (not shown), where the catches 36 release the distal flange 32a and allow the spring 38 to direct the pin sleeve 32 distally to the active position, e.g., sufficient distance such that the puncture pin 34 penetrates the septum 46 of the canister 40.

When the septum 46 is penetrated, gas within the cavity 48 is released into the first chamber 22, e.g., such that the gas travels distally around the canister 40, through the intermediate passage 28, and into the second chamber 26. For example, the drive housing 12 and canister body 42 may have corresponding diameters to provide sufficient clearance to allow the gas to travel distally around the canister 40 within the first chamber 22 and enter the intermediate passage 28, as described elsewhere herein.

Optionally, the intermediate passage 28 may have a relatively small diameter to provide a restrictor to reduce the pressure rise time. Alternatively, a precision orifice (not shown) may be inserted between the first and second chambers 22, 26, if desired to act as a restrictor. For example, an orifice may i) slow down the transient flow of gas, slowing the rise of pressure imparted to the plunger 50, e.g., providing a soft-start to the injection, reducing/eliminating pressure shock waves in the fluid to be injected in the syringe and possibly reducing patient pain as the drug injection is gently initiated; and/or ii) slow down the steady state flow of gas, reducing the otherwise pressure imparted to the plunger 50, providing a limiting effect to the flow rate of the drug injected into the patient.

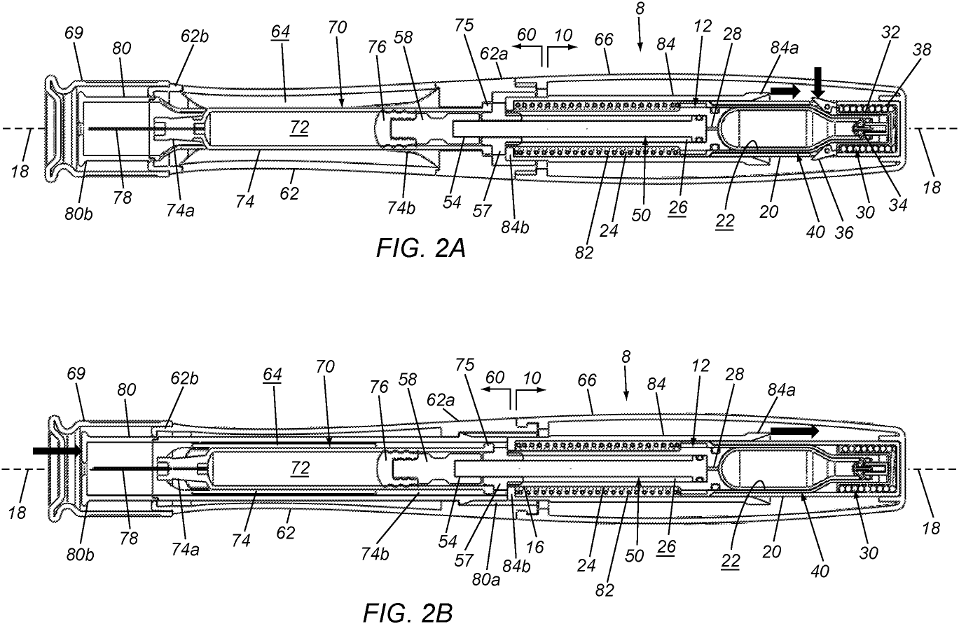

Returning to FIGS. 1-2B, the injector 60 generally includes an injector housing 62 coupled to the drive housing 12 and carrying a syringe 70 therein including an agent chamber 72 containing one or more medicaments or other agents. For example, the injector housing 62 may include a first or proximal end 62a that may be coupled to the drive housing 12, e.g., using one or more of an interference fit, one or more cooperating connectors, bonding with adhesive, and the like, e.g., to an outer sleeve 66 within which at least a portion of the drive housing 12 may be secured, and a second or distal end 62b opposite the drive housing 12. In one example, the proximal end 62a of the injector housing 62 communicates with an interior 64 of the injector housing 62 and the distal end 62b is at least partially enclosed.

Generally, the syringe 70 includes a barrel 74 and a piston or stopper 76 slidably disposed therein to enclose the agent chamber 72. A needle 78 may extend from a closed distal end 74a of the barrel 74. In one example, the syringe 70 may be a pre-filled syringe, e.g., formed from glass, plastic, and the like, filled with a predetermined volume of agent, e.g., corresponding to a single dose for a patient. The agent chamber 72 may include one or more therapeutic and/or diagnostic agents, e.g., a viscous fluid having a viscosity greater than water, e.g., between about one and two thousand centipoise (1.0-2000 cP), e.g., including large proteins and/or other medicaments that require substantial force and/or time to deliver.

Optionally, one or more flanges or other features 75 may be provided on a proximal end 74b of the barrel 74 that may engage one or more detents, ridges, or other features (not shown) within the injector housing 62. For example, during manufacturing or assembly, a syringe 70 may be selected that may be inserted into the interior 64 through the proximal end 62a of the injector housing 62, e.g., until the needle 78 extends partially through the distal end 62b and the flange 75 on the syringe 70 is captured by the feature(s) on the injector housing 62. The proximal end 62a of the injector housing 62 may then be coupled to the outer sleeve 66 of the drive module 10 to encapsulate the components and provide the injector device 8 ready for use.

Alternatively, the injector module 60 may include an integral barrel (not shown) defining the agent chamber 72 and carrying the needle 78. For example, the injector housing 62 may define a substantially enclosed agent chamber (not shown) that slidably receives the piston 76 and includes a needle 78 permanently mounted to the injector housing 62 for delivering the agent within the agent chamber 72. In a further alternative, the syringe 70 (or injector housing 62 with integral agent chamber) may include a distal port (not shown) without a needle, such that a separate needle (also not shown) may be coupled to the port, e.g., using a Luer fitting, mating threads, and/or other cooperating connectors, immediately before an injection or otherwise as desired.

The piston 76 may be coupled to the distal end 54 of the plunger 50, e.g., during assembly of the injector housing 62 to the outer sleeve 66, such that subsequent advancement of the plunger 50 causes the piston 76 to advance within the agent chamber 72 to direct the one or more agents through the needle 78 into a patient’s body, e.g., automatically upon activation of the puncture mechanism 30, as described further elsewhere herein. Optionally, a plunger adapter 58 may be provided that may provide an interface between the distal end 54 of the plunger 50 and the piston 76, e.g., to provide connectors therebetween and/or ensure proper spacing such that the piston 76 is advanced in conjunction with the plunger 50.

The plunger 50 may be an elongate rod or other member including a proximal end 52 and a distal end 54 that is slidably disposed within the second chamber 26 such that the plunger 50 that is movable from an initial or retracted position (e.g., shown in FIG. 3A) to a final or extended position (e.g., shown in FIG. 3B) wherein the distal end 54 extends from the second end 16 of the drive housing 12. A piston or other seal 53, e.g., including one or more O-rings, is provided on the proximal end 52 of the plunger 50 that slidably engages a wall of the second chamber 26.

During use, the drive module 10 may be coupled to an injector module or other delivery device (or already integrated into such a device), and used to deliver one or more agents within an agent housing of the delivery device (not shown). For example, the injector module may house a syringe including an injection piston in its proximal end and a needle on its distal end and containing a preset volume of agent for delivery, similar to the devices disclosed in U.S. Pat. No. 11,071,824.

Once the delivery device is positioned as desired, e.g., inserting the needle into a target location of the subject’s body, an actuator of the delivery device may be activated to cause the puncture pin 34 to penetrate the septum 44 of the canister 40, thereby causing pressurized gas from the canister 40 to pass through the first chamber 22 and intermediate passage 28 into the second chamber 26. The resulting pressure applies a distal force to the piston 53, causing the plunger 50 to advance from the initial position (FIG. 3A) to the extended position (FIG. 3B). Alternatively, the needle may be initially spaced away from the patient’s skin when the distal end is placed against the skin, and activation may also advance the needle to puncture the skin and deliver the agent and/or retract the needle after delivery, similar to injectors in U.S. Pat. No. 11,071,824.

As can be noted from FIGS. 3A and 4A, when the septum 44 is initially punctured, the initial volume that the gas must fill (including the first chamber 22 around the canister 40 and the second chamber 26 proximal to the piston 53) is relatively small. However, as the plunger 50 advances, the volume within the second chamber 26 that the gas must fill increases substantially. Because the canister 40 has a fixed initial pressure (set when it is filled), as the escaping gas fills the increasing volume, the pressure decreases substantially. Because the distal force applied by the gas on the piston 53 is proportional to this pressure, as the plunger 50 moves distally, the force applied to the plunger 50 also decreases substantially. This resulting force drop may cause the plunger 50 to slow as it advances, which can slow delivery time and/or result in incomplete delivery of the agent, particularly if the agent being delivered is viscous, which can exacerbate the force drop.

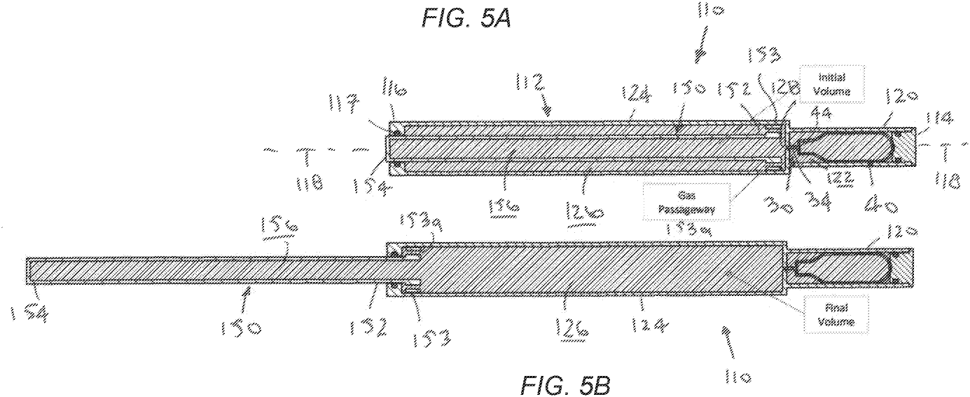

Turning to FIGS. 5A and 5B, another example of a drive module 110 is shown that maybe included in an injector device, such as the device 8, which may substantially reduce the force drop compared to the drive module 10. Similar to the drive module 10, the drive module 110 includes an elongate drive housing 112 containing a puncture mechanism 30 including a puncture pin 34, a gas canister or other source of pressurized gas 40, and a plunger 150. The housing 112 also includes a first or proximal portion 120 adjacent the first end 114 defining a first chamber 122 for receiving the canister 40 and puncture mechanism 30, and a second or distal portion 124 adjacent the second end 116 defining a second chamber 126 communicating with the first chamber 122 via an intermediate passage 128, all of which may be constructed generally similar to the drive module 10.

The puncture mechanism 30 and canister 40 may be provided within the first chamber 122 adjacent the intermediate passage 128 with the septum 44 of the canister 40 spaced apart initially from the puncture pin 34, e.g., oriented distally as shown or oriented proximally (not shown), also similar to the drive module 10. An actuator may be provided that may move one or both of the puncture mechanism 30 and/or canister 40 to cause the puncture pin 34 to penetrate the septum 44 and release the gas, similar to the drive module 10 and the devices disclosed in U.S. Pat. No. 11,071,824, incorporated by reference herein.

The plunger 150 may be an elongate rod or other member including a proximal end 152 and a distal end 154 that is slidably disposed within the second chamber 126 such that the plunger 150 is movable from an initial or retracted position (e.g., shown in FIG. 5A) to a final or extended position (e.g., shown in FIG. 5B) wherein the distal end 154 extends from the second end 116 of the drive housing 112.

Unlike the drive module 10, however the plunger 150 includes a piston 153 on the proximal end 152 that includes one or more passages 153a that extend between proximal and distal surfaces of the piston 153. For example, the piston 153 may include a plurality of circular or other enclosed passages 153a spaced apart from one another around a circumference of the piston 153. Alternatively, the passage(s) may be grooves formed in the outer surface of the piston 153 (not shown) that extend between the proximal and distal surfaces. The piston 153 may be sized and/or shaped to slidably engage a wall of the second chamber 126, e.g., to allow the plunger 150 to move from the initial to the extended position, but does not require O-rings or other seals. For example, the piston 153 may be a cylindrical head having a larger outer diameter than the plunger 150 that is integrally molded or otherwise formed with the plunger 150, or that is manufactured separately and permanently attached to the plunger 150.

As can be seen in FIG. 5A, the passage(s) 153a communicate between the region of the second chamber 126 proximal to the piston 153 and the region distal to the second chamber 126 surrounding plunger 150. The second end 116 of the housing 112 may include one or more O-rings or other seals 117 that may slidably engage the plunger 150, i.e., to provide a fluid-tight seal without interfering substantially with axial movement of the plunger 150.

Optionally, the plunger 150 may also include a plunger chamber 156, e.g., extending from the open proximal end 152 of the plunger 150 to the closed distal end 154. Consequently, when the septum 44 of the canister 40 is initially penetrated, gas within the canister 40 may pass freely from the first chamber 122 through the intermediate passage 128 and into the second chamber 126, i.e., through the passage(s) 153a into the second chamber 126 around the plunger 150 and into the plunger chamber 156, as described further below.

During use, the drive module 110 may be coupled to an injector module or other delivery device (or already integrated into such a device) and used to deliver one or more agents within an agent housing of the delivery device (not shown). For example, the drive module may be coupled to an injector module housing a syringe including a piston in its proximal end and a needle on its distal end and containing a preset volume of agent for delivery, similar to the devices disclosed in U.S. Pat. No. 11,071,824.

Once the delivery device is positioned as desired, e.g., after inserting the needle into a target location of a subject’s body (or placing the auto-injector against the skin), an actuator the of the delivery device may be activated to cause the puncture pin 34 to penetrate the septum 44 of the canister 40, thereby causing pressurized gas from the canister 40 to pass through the first chamber 122 and intermediate passage 128 into the second chamber 126. As with the drive module 10, the resulting pressure applies a distal force to the plunger 150, causing the plunger 150 to advance from the initial position (FIG. 5A) to the extended position (FIG. 5B).

However, as can be noted from FIG. 5A, when the septum 44 is initially punctured, the initial volume that the gas must fill (including the first chamber 122 around the canister 40, the second chamber 126 around the plunger 150 and, optionally, the plunger passage 156) is substantially larger than the drive module 10. This may result in an initial pressure drop; however, as the plunger 150 advances, the change in volume that the gas must fill increases only minimally (e.g., the volume the plunger 150 occupies within the second chamber 126 that is displaced out of the distal end 116 of the housing 112). Consequently, because the volume change is minimized, the resulting force applied by the pressure on the plunger 150 may remain substantially constant or reduce only slightly, particularly as compared to the drive module 10. Thus, the resulting force drop applied to the plunger 150 may be minimized, which may provide a more uniform delivery rate of the agent from the delivery device, particularly if the agent being delivered is viscous.

Although the drive module 110 includes a canister and puncture mechanism as the source of pressurized gas, it will be appreciated that the drive module 110 may include other sources of pressurized gas that may be actuated to release the pressurized gas into the second chamber 126, i.e., around and/or into the chamber 156 of the plunger 150, to advance the plunger 150 with minimized force drop. For example, an external canister or gas source may be connected to the drive module 110 before use, or the gas canister may include arrangements other than a septum and puncture pin to release the gas from the canister.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A drive module for an injection device for delivering one or more agents into a subject’s body, comprising:
an elongate drive housing including a first end and a second end, and a chamber;
a source of pressurized gas communicating with a passage into the chamber;
a plunger slidably disposed within the chamber comprising a proximal end including a piston and a distal end, the plunger configured such that gas entering the chamber from the passage causes the plunger to move from an initial position to an extended position wherein the distal end of the plunger extends from the second end of the drive housing for delivering one or more agents from an injector module based on movement of the plunger,
wherein the piston comprises one or more passages configured to allow gas from the canister entering the chamber to pass through the piston into the chamber around the plunger.

2. The drive module of claim 1, wherein the plunger comprises a plunger chamber extending from an opening in the proximal end communicating with the second such that pressurized gas from the canister entering the chamber fills the plunger chamber.

3. The drive module of claim 1, further comprising an actuator for opening a path from the source of pressurized gas through the passage into the chamber.

4. The drive module of claim 1, wherein the source of pressurized gas comprises:
a puncture mechanism comprising a pin holder within the drive housing adjacent the first end including a puncture pin;
a canister containing pressurized gas including a penetrable septum disposed adjacent the puncture pin, the pin holder movable from an inactive position wherein the puncture pin is spaced away from the septum and an active position wherein the puncture pin penetrates the septum and causes the gas within the canister to flow through the passage into the second chamber; and
an actuator for directing the pin holder from the inactive position to active position.

5. The drive module of claim 4, wherein the actuator comprises:
one or more catches on the drive housing adjacent the pin holder for restraining the pin holder in the inactive position;
an actuation sleeve slidably disposed over the drive housing and comprising a proximal end disposed distal to the one or more catches such that, movement of the actuation sleeve proximally disengages the one or more catches, whereupon the pin holder automatically moves from the inactive position to the active position to activate the drive module.

6. The drive module of claim 4, wherein the pin holder comprises a lead screw comprising threads that cooperate with the first end of the drive housing such that rotation of the lead screw causes the lead screw to move from the inactive position to the active position.

7. The drive module of claim 6, wherein the actuator comprises a rotating handle mounted on the first end of the drive housing and coupled to the lead screw, the handle rotatable relative to the drive housing for directing the lead screw from the inactive position to the active position.

8. The drive module of claim 4, wherein the actuator comprises a handle pivotally coupled to the drive housing and a linking bar coupled between the handle and the pin holder such that rotation of the handle about a pivot to cause the linking bar to direct the pin holder from the inactive position to the active position.

9. The drive module of claim 4, wherein the actuator comprises an actuation button extending from the drive housing and coupled to the pin holder, the actuation button advanceable to direct the pin holder distally for sufficient distance to move the pin holder from the inactive position to the active position.

10. The drive module of claim 4, wherein the septum of the canister is oriented proximally within the drive housing, and the puncture mechanism is disposed proximal to the canister such that the puncture pin is oriented distally towards the septum such that distal movement of the pin holder causes the puncture pin to move distally to penetrate the septum.

11. The drive module of claim 4, wherein the septum of the canister is oriented distally within the drive housing, and the puncture mechanism is disposed distal to the canister such that the puncture pin is oriented proximally towards the septum such that proximal movement of the pin holder causes the puncture pin to move proximally to penetrate the septum.

12. (canceled)

13. A device for delivering one or more agents into a patient’s body, comprising:
a) a drive module comprising:
an elongate drive housing including a first end and a second end, a first chamber adjacent the first end communicating with a second chamber adjacent the second end via an intermediate passage;
a puncture mechanism within the first chamber adjacent the first end including a puncture pin;
a canister containing pressurized gas including a penetrable septum disposed adjacent the puncture pin;
an actuator configured to move one of the puncture mechanism and the canister to cause the puncture pin to penetrate the septum and cause the gas within the canister to flow through the first chamber around the canister, through the intermediate passage, and into the second chamber; and
a plunger slidably disposed within the second chamber comprising a proximal end including a piston and a distal end, the plunger configured such that gas entering the second chamber from the passage causes the plunger to move from an initial position to an extended position wherein the distal end of the plunger extends from the second end of the drive housing for delivering one or more agents from an injector module based on movement of the plunger; and
b) an injector module comprising:
an injector housing coupled to the drive housing carrying an agent chamber containing one or more agents;
a piston slidably disposed within the agent chamber and coupled to the distal end of the plunger; and
a needle extending from the injector module opposite the drive housing and communicating with the agent chamber for delivering the one or more agents from the agent chamber when the plunger moves from the retracted position to the extended position, thereby advancing the piston within the agent chamber,
wherein the piston comprises one or more passages configured to allow gas from the canister entering the first chamber to pass through the piston into the chamber around the plunger.

14. The device of claim 13, wherein the plunger comprises a plunger chamber extending from an opening in the proximal end communicating with the second such that pressurized gas from the canister entering the second chamber fills the plunger chamber.

15. The device of claim 13, wherein the injector further comprises a needle guard movable from a guarded position wherein the needle guard covers the needle and a retracted position wherein the needle is exposed to perform an injection, the needle guard coupled to the actuator and slidable along the drive housing such that the needle guard activates the actuator when directed to the retracted position.

16. The device of claim 15, wherein the needle guard is biased to the guarded position.

17. The device of claim 16, further comprising:
an actuation sleeve slidably disposed over the drive housing and coupled to the needle guard; and
a spring coupled between the actuation sleeve and drive housing to bias the needle guard to the guarded position.

18. The device of claim 13, wherein the puncture mechanism comprises a pin sleeve slidably disposed within the first chamber adjacent the first end and carrying the puncture pin, the pin sleeve movable from an inactive position wherein the puncture pin is spaced apart from the septum and an active position wherein the puncture pin penetrates the septum, the pin sleeve biased to the active position and restrained in the inactive position by the actuator.

19. The device of claim 18, wherein the actuator comprises one or more catches on the drive housing restraining the pin sleeve in the inactive position, the actuation sleeve configured to disengage the one or more catches to allow the pin sleeve to automatically move from the inactive position to the active position.

20-34. (canceled)

35. A method for performing an injection, comprising:
providing an injection device according to claim 1;
inserting the needle through a patient’s skin; and
activating an actuator to deliver pressurized gas from the source through the passage into the chamber, thereby moving the plunger from the initial position to the extended position and, consequently, advancing the piston within the agent chamber to deliver the one or more agents through the needle into the patient’s body.

36-41. (canceled)
